(19) United States
(12) Patent Application Publication
(10) Pub. No.: US 2023/0110013 A1
(43) Pub. Date: Apr. 13, 2023

(54) COMPOSITIONS AND METHODS PROVIDING NUTRITIONAL AND OTHER BENEFITS HAVING ANTIVIRAL PROPERTIES

(71) Applicant: Furst-McNess Company, Freeport, IL (US)

(72) Inventor: Fredrik B. SANDBERG, Johnston, IA (US)

(21) Appl. No.: 17/943,646

(22) Filed: Sep. 13, 2022

Related U.S. Application Data
(63) Continuation-in-part of application No. PCT/US21/22320, filed on Mar. 15, 2021.
Application No. PCT/US21/22320 is a continuation-in-part of application No. PCT/US21/22331, filed on Mar. 15, 2021.
(60) Provisional application No. 62/989,293, filed on Mar. 13, 2020.
(60) Provisional application No. 63/005,844, filed on Apr. 6, 2020.
(60) Provisional application No. 63/064,574, filed on Aug. 12, 2020.
(60) Provisional application No. 62/989,293, filed on Mar. 13, 2020.
(60) Provisional application No. 63/005,844, filed on Apr. 6, 2020.
(60) Provisional application No. 63/064,574, filed on Aug. 12, 2020.

(51) Int. Cl. A61K 31/22; A61K 36/9066; A61K 31/122; A61K 31/05; A23K 10/00; A61K 36/534

(52) U.S. Cl. CPC A61K 31/22; A61K 36/9066; A61K 31/122; A61K 31/05; A23K 10/00; A61K 36/534

(57) ABSTRACT

Compositions and methods having antiviral properties, such as for the prevention, treatment or providing nutritional or other benefits in animals, including humans and livestock, having or at risk of viral-mediated disorders. Methods may provide nutritional benefits in a human or animal subject, comprising administering a composition of the present invention. Compositions may comprise monoglycerides and essential oils, including compositions comprising two or more of the following components: a monoglyceride, a curcuminoid, thymoquinone, thymol, carvacrol, and p-cymene. Such viral mediated disorders may be associated with viruses of the family Coronaviridae (e.g., MERS-CoV, SARS-CoV, SARS-CoV-2), viruses of the family Orthomyxoviridae (e.g., Influenza A, Influenza B), Porcine Epidemic Diarrhea Virus, Transmissible Gastroenteritis, Porcine Respiratory Reproductive Syndrome, African Swine Fever Virus, Foot-and-Mouth Virus, Seneca Valley Virus, Influenza A, Avian Influenza H5N1, Rotavirus A, Rotavirus B, Rotavirus C, or Rotavirus D.

### CROSS-REFERENCE TO RELATED APPLICATIONS

This application is a continuation-in-part of PCT International Application PCT/US2021/022320 filed on Mar. 15, 2021, which claims the benefit and priority of U.S. Provisional Application 63/064,574 filed on Aug. 12, 2020, U.S. Provisional Application 63/005,844 filed on Apr. 6, 2020, and U.S. Provisional Application 62/989,293 filed on Mar. 13, 2020. This application is also a continuation-in-part of PCT International Application PCT/US2021/022331 filed on Mar. 15, 2021, which also claims the benefit and priority of U.S. Provisional Application 63/064,574 filed on Aug. 12, 2020, U.S. Provisional Application 63/005,844 filed on Apr. 6, 2020, and U.S. Provisional Application 62/989,293 filed on Mar. 13, 2020. The entire disclosures of each of the above applications are incorporated herein by reference.

### BACKGROUND

The present technology relates to compositions and methods having antiviral properties, such as for the prevention, treatment or providing nutritional or other benefits in animals, including humans and livestock, having or at risk of viral-mediated disorders. In various embodiments, the compositions may be nutritional, nutraceutical or pharmaceutical.

Viruses cause many known diseases, including the common cold and flu. In many instances, such diseases have limited effect on infected individuals. However, viral diseases may result in significant morbidity or mortality, particularly in individuals having compromised immune systems or other physical conditions (e.g., diabetes) that may complicate their ability to overcome the viral infections or secondary infections. For example, viral infections may cause aberrant immune system responses, even in individuals that are otherwise healthy. Such aberrant responses may include hypercytokinemia (a “cytokine storm”) or cytokine release syndrome associated with overproduction of immune cells and inflammatory cytokines, which can cause multisystem organ failure and death. In livestock, viruses lead to sickness, loss of growth performance, and reduced efficiency of converting feed stuffs into meat, as well as death loss and impaired welfare of livestock.

A hallmark of viral-mediated diseases is the continual evolution of the virus infective agents, as seen in annual variations, which can complicate their control and treatment. Public health consequences can be particularly acute for diseases caused by unexpected and rapid development of novel viruses that have the ability to infect individuals across species, for example, crossing from bats or other animals to humans, as seen in diseases such as Ebola virus disease (EVD) and severe acute respiratory disease (SARS). This evolution presents significant challenges in the control of virus-mediated diseases in humans and other animals, as vaccines and acquired immunity to previous virus exposure may have no protective effect, and treatments useful against previous infective agents may be ineffective.

Many materials such as organic acids, medium chain fatty acids and glycerides, essential oils, phenols, alkaloids amongst others have been tested and suggested to have efficacy in vitro for destroying viruses. However, the utility of such materials in actual in vivo methods of preventing or treating viral infections may be uncertain, depending on the ability of such agents to be effectively delivered, their actual efficacy in vivo, and the potential for toxicity.

Accordingly, there is an acute need for new nutritional, preventative and therapeutic modalities that mediate the effects of virus infections in humans and other animals. For example, there is a need for compositions and methods that prevent, treat or reduce the negative physiological effects of infections in humans from viruses such as Influenza A (e.g., H1N1), Influenza B, Ebola virus, Norovirus, Rotavirus, SARS-CoV, SARS-CoV-2 (the causative agent for COVID-19), and other known or novel viral infective agents. Similarly, there is a need to develop compositions and methods prevent, treat or reduce the negative physiological effects of infections in livestock from viruses such as PEDv, PRRSv, SVA, ASFv, as well as other known and novel viral infective agents. Preventing negative outcomes from such infections in livestock can have significant economic benefits by improving livestock welfare, performance, and survivability.

### SUMMARY

The present technology provides compositions and methods providing antiviral benefits, such as for the prevention, treatment or providing nutritional benefits in human or other animal subjects having, or at risk of having, viral-mediated disorders. In various aspects, such compositions and methods are useful in the prevention, treatment, or amelioration of viral infections in animals, including humans and livestock. Such compositions and methods may be nutritional, nutraceutical or pharmaceutical.

In various embodiments, the present technology provides compositions comprising monoglycerides and essential oils, including compositions comprising two or more of the following components: a monoglyceride, a curcuminoid, thymoquinone, thymol, carvacrol, and p-cymene. Compositions may comprise three or more, or four or more of the components. In some embodiments, compositions comprise a monoglyceride, a curcuminoid, thymoquinone, thymol, and carvacrol. In some embodiments, compositions comprise monolaurin, curcuminoids and thymoquinone.

Accordingly, the present technology provides formulations comprising one or more monoglycerides (such as alpha monoglycerides) comprising short chain carboxylic acids such as formic acid, acetic acid, propionic acid, butyric acid or valeric acid, medium chain carboxylic acids such as caproic acid, caprylic acid, capric acid and long chain carboxylic acids such as lauric acid, myristic acid, pentadecanoic acid and hexadecenoic acid.

In various embodiments compositions of the present technology comprise curcuminoids, which may be derived from or provided by turmeric, or manufactured synthetically. Curcuminoids may be present as an extract, as part of whole standardized turmeric and with or without tumerones. Compositions may comprise thymoquinone, which may be derived from or provided by black cumin seed, Monarda fistulosa, or manufactured synthetically. Compositions may comprise thymol, which may be derived from or provided by Monarda fistulosa, essential oil of thyme, or manufactured synthetically. Compositions may comprise carvacrol, which may be derived from or provided by Monarda fistulosa, essential oil of oregano, or manufactured synthetically.

The present technology also provides methods for mediating a nutritional or physiological status of a human or animal subject by administering a composition of the present technology. In various aspects, such methods comprise administering to the subject a composition comprising: a monoglyceride, a curcuminoid, thymoquinone, thymol, and carvacrol. In some embodiments, methods are for maintaining or improving the nutritional status of a human or animal subject. The subject may have, or is at risk of having, a viral-mediated disorder. Such viral mediated disorders may be associated with viruses of the family Coronaviridae (e.g., MERS-CoV, SARS-CoV, SARS-CoV-2), or viruses of the family Orthomyxoviridae (e.g., Influenza A, Influenza B). In some embodiments, such viral-mediated disorders are associated with Porcine Epidemic Diarrhea Virus, Transmissible Gastroenteritis, Porcine Respiratory Reproductive Syndrome, African Swine Fever Virus, Foot-and-Mouth Virus, Seneca Valley Virus, Influenza A, Avian Influenza H5N1, Rotavirus A, Rotavirus B, Rotavirus C, or Rotavirus D.

In some embodiments, methods provide nutritional benefits in a human or animal subject, comprising administering a composition of the present invention. For example, such nutritional benefits may be selected from the group consisting of maintaining, strengthening or improving immune function, maintaining, strengthening or improving immune status, maintaining, strengthening or improving immune health, maintaining, strengthening or improving innate immune function, maintaining, strengthening or improving respiratory function, reducing the duration of symptoms associated with respiratory disease, reducing the severity of symptoms associated with respiratory disease, and combinations thereof. Further, in various embodiments, methods provide cognitive benefits, such as strengthening or improving cognitive function, strengthening or improving mental focus, strengthening or improving mental acuity, strengthening or improving memory, strengthening or improving functional capacity, and strengthening or improving emotional well-being, strengthening or improving endurance performance, strengthening or improving endurance capacity, strengthening or improving alertness, strengthening or improving concentration, providing energy, and reducing fatigue.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

### Compositions

As previously discussed, the present technology provides compositions suitable for administration to animals, for example humans, livestock and other animals. Such compositions comprise components that are acceptable for the intended administration to human or animal subjects in methods of the present technology. In general, such physiologically-acceptable components provide the intended benefit in the subject to whom the compositions are administered, without undue adverse side effects (such as toxicity, irritation, or allergic response), commensurate with a reasonable benefit/risk ratio when used in the manner of this technology. In various embodiments, such physiologically-acceptable components are “nutritionally-acceptable” components, providing a nutritional benefit. In other aspects, such physiologically-acceptable components are “pharmaceutically-acceptable,” providing an intended pharmaceutical benefit. In various embodiments, compositions comprise components that are generally recognized as safe (GRAS), as may be designated by the United States Food and Drug Administration or other applicable regulatory authority.

### Monoglycerides

The compositions of the present technology comprise monoglycerides (acylglycerols, or monoacylglycerols). Monoglycerides include alpha monoglycerides (1-monoacylglycerols) and beta monoglycerides (2-monoacylglycerols), wherein the fatty acid is linked to the sn-1 position of glycerol via an ester bond. In some preferred embodiments, monoglycerides comprise, or are, alpha monoglycerides. In various embodiments, monoglycerides, are used herein in admixture with diglycerides (preferably below about 20% by weight of mixture]) and triglycerides (preferably below about 10% by weight of mixture. Such monoglycerides comprise glycerol linked to a carboxylic acid (e.g., fatty acid) through an ester bond. Such acids may be short chain acids (having a carbon chain of from one to five carbons, C1-C5), medium chain acids (C6-C12) or long chain acids (C13-C20), or combinations thereof. Short chain carboxylic acids include formic acid (C1), acetic acid (C2), propionic acid (C3), butyric acid (C4) and valeric (C5) acid. Medium chain carboxylic acids include caproic acid (C6), caprylic acid (C7), and capric acid (C10). Long chain carboxylic (fatty) acids include lauric acid (C12), myristic acid (C14), pentadecanoic acid (C15) and hexadecenoic acid (C16).

In some embodiments, compositions comprise monoglycerides of carboxylic acids, but some optional components may include some proportion of carboxylic acids in their original acid form (e.g. lauric acid as opposed to monolaurin). For example, compositions may comprise a blend of mono- and di-glycerides of propionic acid (monopropionine), butyric acid (monobutyrin), caproic acid (monocaproin), caprylic acid (monocaprylin), capric acid (monocapricin) and lauric acid (monolaurin), but may include mono- and di-glycerides from other carboxylic acids. These may be produced from carboxylic acids derived from palm oil, coconut oil, and other base oils or fat-forming acids.

In various embodiments, compositions of the present technology comprise a monoglyceride selected from the group consisting of alpha-monoglyceride of monopropionine (2,3-dihydroxypropyl propionate; molecular weight 148.16), alpha-monoglyceride of monobutyrin (2,3-dihydroxypropyl butanoate; molecular weight 162.18), alpha-monoglyceride of monocaprylin (2,3-dihydroxypropyl octanoate; molecular weight 218.29), alpha-monoglyceride of monocaprin (2,3-dihydroxypropyl decanoate; molecular weight 246.34) and alpha-monoglyceride of monolaurin (2,3-dihydroxypropyl dodecanoate; 1-lauroyl-glycerol; or glycerol monolaurate; molecular weight 274.4), and mixtures (blends) thereof. In some embodiments, compositions comprise monoglycerides that comprise, or are, monolaurin, and which may also comprise di-laurin (diglyceride form) and tri-laurin (triglyceride form). Alpha-monoglyceride of monopropionine (a C3 acid) is classified as a preserving agent for animal feed and grains. In some embodiments, the monoglyceride comprises, or is, monolaurin.

### Botanical Components

The compositions of the current technology may comprise one or more essential oils, oleoresins or other botanical components, it being understood that such botanical components may be naturally provided or derived, or manufactured synthetically. In various embodiments, the compositions comprise botanical components in the form of herbs, spices, essential oils or other extracts thereof, and combinations thereof. Such extracts and compounds may be isolated from natural sources using methods among those known in the art, such as disclosed in Douglas, et al., Herbs, Spices and Essential Oils, UNIDO and FAO 2005.

In various embodiments, compositions of the present technology comprise a botanical selected from the group consisting of curcuminoids, thymoquinone, thymol, carvacrol, p-cymene, and mixtures thereof. In various embodiments, curcuminoids (CI Natural Yellow 3; (1E,6E)-1,7-bis(4-hydroxy-3-methoxyphenyl)hepta-1,6-diene-3,5-dione, molecular weight 368.4) can be provided as turmeric, or extracted from the root of the turmeric plant, or can be produced synthetically. Thymol (5-methyl-2-propan-2-ylphenol; 1-methyl-3-hydroxy-4-isopropylbenzene, molecular weight 150.22) can be provided by or extracted from the essential oil of the Thyme plant (Thymus vulgaris), the Monarda plant (e.g., Monarda fistulosa), or oregano oil, or it can be produced synthetically. Carvacrol (2-methyl-5-propan-2-ylphenol; 2-hydroxy-p-cymene, molecular weight 150.22) can be provided, or extracted, from the essential oil of Monarda, or from the essential oil of oregano (Origanum vulgare), or it can be produced synthetically. Thymoquinone (2-methyl-5-propan-2-ylcyclohexa-2,5-diene-1,4-dione, molecular weight 164.2) can be provided or extracted from the essential oil of Monarda, or can be provided or extracted from black cumin seeds, or it can be produced synthetically. P-cymene(1-isopropyl-4-methylbenzene, molecular weight 134.22) can be provided or extracted from the essential oil of the Thyme plant (Thymus vulgaris), from the essential oil of oregano, from the essential oil of Eucalyptus, from the essential oil of Monarda, or can be produced synthetically.

In various embodiments, compositions comprise botanical materials or extracts that comprise the botanical components of the present compositions. For example, compositions may comprise oregano essential oils, derived from the leaves and flowers of the herb, Origanum vulgare, which comprises carvacrol and thymol. In some embodiments, compositions comprise an extract from Monarda fisulosa, which comprises thymoquinone, thymol, carvacrol, and p-cymene. Accordingly, the present technology provides compositions comprising: a monoglyceride (an alpha-monoglyceride), a curcuminoid, thymoquinone, thymol, carvacrol, and p-cymene.

As discussed further below, the compositions of the present technology may be formulated in products comprising the components of the present compositions and optional physiologically-acceptable materials. Such optional materials may be operable as a carrier for administration of the compositions to a human or animal subject. Products may be liquid, solid or semi-solid depending on the components of the composition and the desired method of administration (which may be further dependent on the benefit to be provided). Such carriers may be aqueous. Formulation aids may include emulsifiers and thickeners. For example, emulsifiers may include soy and egg lecithin, mono- and diglycerides, polysorbates (e.g., polysorbate 80), carrageenan, guar gum, and canola oil.

Compositions and products of the present technology may comprise optional components, including additional nutritional components (such as optional botanicals, vitamins, minerals and amino acids), absorption enhancing components, digestive aids (such as enzymes and probiotics), flavorants (artificial and natural flavor components), preservatives, and formulation aids. For example, optional botanicals include rosemary (source of rosmarinic acid), garlic (source of allicin), cinnamon (source of cinnamaldehyde) and olive leaf extract (source of oleuropein).

### Formulations

In general, the components of the present compositions are used in the compositions and methods of the present technology at safe and effective levels. Such levels may be “physiologically-acceptable,” providing a nutritional or other physiological or pharmacological benefit to the human or animal subject, without undue adverse side effects (such as toxicity, irritation, or allergic response in the subject, or in a human ingesting or otherwise using products derived from a livestock subject), commensurate with a reasonable benefit/risk ratio when used in the manner of this technology. For example, components may be used at nutritionally safe and effective levels in the compositions and methods of the present technology, i.e., at levels that are effective to provide a nutritional or other physiological benefit to the subject, without undue adverse side effects (such as toxicity, irritation, or allergic response in the subject or in a human ingesting or otherwise using products derived from a livestock subject), commensurate with a reasonable benefit/risk ratio when used in the manner of this technology.

Without limiting the mechanism, function or utility of present technology, in various embodiments, the compositions of the present invention comprise two or more components that afford synergistic benefits in the prevention, treatment or providing nutritional or other benefits, such as benefits relating to viral-mediated disorders in animals, present at synergistic levels. As referred to herein, such synergistic combinations comprise components at levels that, when administered to (e.g., ingested by) a human or other animal, afford a nutritional, physiological, pharmacological or other benefit greater than the additive effect of administering each component individually. In various aspects, individual components of the compositions may provide different and specific functions in vivo within the body of the subject to whom the compositions are administered. In various embodiments, compositions comprising a combination of components may provide benefits at low levels of administration, which may offer enhanced safety and reduced risk of toxicity relative to compositions containing each component individually.

In various embodiments, wherein the curcuminoid is a component of an extract, the extract being present in the composition at a level of from about 0.1% to about 5%, or from about 0.2% to about 2%, or from about 0.5% to about 1%. Thymoquinone may be present at a level of from about 0.01% to about 0.2%. Thymol may present at a level of from about 0.01% about 0.2%. Carvacrol is present at a level of from about 0.01% about 20%. P-Cymene may be present at a level of from about 0.01% to about 30%.

Compositions of the present technology may comprise a Monarda extract, wherein the Monarda extract comprises thymoquinone, thymol, carvacrol, and p-cymene. For example, the Monarda extract may be present at a level of from 0.1% to about 5%, or from about 0.2% to about 2%, or from about 0.5% to about 1%. For example, compositions may comprise from about 15% to about 25% of monoproprionine; from about 15% to about 25% of monobutyrin; from about 10% to about 15% of monocaprin; from about 10% to about 15% of monocaproic; from about 10% to about 20% of monolaurin; from about 0.5% to 5% of an extract comprising pure curcuminoids (e.g., having a concentration of about 95% to 99% of curcuminoids); from about 0.5% to about 5% of a Monarda extract; from about 1% to about 10% of surfactant (e.g., polysorbate 80); and 1 to 10% co-surfactant/emulsifier (e.g. lecithin) and from about 20% to 50% of water.

In some embodiments, aqueous compositions comprise an emulsion (e.g., oil-in-water) of components. The emulsion may be a microemulsion or a nanoemulsion, and may be characterized as a colloidal delivery system. In general, such emulsions comprise an oil or lipid phase (e.g., comprising water insoluble or poorly soluble monoglycerides), an aqueous phase (e.g., comprising water soluble monoglycerides, Monarda extract and other extracts providing other components of the present compositions), and one or more emulsifiers (e.g., a polysorbate, such as polysorbate 80 or other nonionic emulsifier). Particles in the emulsion may have a mean or median size (radius) of less than about 100 nm. Such emulsions are generally described in McClements, Nanoemulsions versus microemulsions: terminology, differences, and similarities, 8 Soft Matter, 1719 (2012).

Exemplary formulations are set forth in the following tables.

### Example Formula 1

| Component | Level |
|---|---|
| mono- and di-glycerides of propionic acid | 0.01 to 50% (wt) |
| (C3:0) (monopropionine) | |
| mono- and di-glycerides of butyric (C4:0) | 0.01 to 50% (wt) |
| acid (monobutyrin) | |
| mono- and di-glycerides of caproic acid (C6:0) | 0.01 to 50% (wt) |
| acid (monocaprin) | |
| mono- and di-glycerides of caprylic (C8:0) | 0.01 to 50% (wt) |
| acid (monocaprin) | |
| mono- and di-glycerides of capric (C10:0) | 0.01 to 50% (wt) |
| acid (monocapricin) | |
| mono- and di-glycerides of lauric (C12:0) | 0.01 to 50% (wt) |
| acid (monolaurin) | |
| Curcuminoids | 0.1 to 50,000 ppm (wt) |
| Thymol | 0.1 to 50,000 ppm (wt) |
| Carvacrol | 0.1 to 200,000 ppm (wt) |
| Thymoquinone | 0.1 to 50,000 ppm (wt) |

### Example Formula 2

| Component | Level |
|---|---|
| mono- and di-glycerides of formic acid (C1:0) (monoformin) | 0.01 to 50% (wt) |
| mono- and di-glycerides of acetic (C2:0) acid (monoacetin) | 0.01 to 50% (wt) |
| mono- and di-glycerides of propionic acid (C3:0)(monopropionine) | 0.01 to 50% (wt) |
| mono- and di-glycerides of butyric (C4:0) acid (monobutyrin) | 0.01 to 50% (wt) |
| mono- and di-glycerides of valeric (C5:0) acid (monovalerin) | 0.01 to 50% (wt) |
| mono- and di-glycerides of caproic (C6:0) acid (monocaprin) | 0.01 to 50% (wt) |
| mono- and di-glycerides of caprylic (C8:0) acid (monocaprin) | 0.01 to 50% (wt) |
| mono- and di-glycerides of capric (C10:0) acid (monocapricin) | 0.01 to 50% (wt) |
| mono- and di-glycerides of lauric (C12:0) acid (monolaurin) | 0.01 to 50% (wt) |
| Curcuminoids | 0.1 to 250,000 ppm (wt) |
| Thymol | 0.1 to 50,000 ppm (wt) |
| Carvacrol | 0.1 to 250,000 ppm (wt) |
| Thymoquinone | 0.1 to 50,000 ppm (wt) |
| Rosemarinic acid | 0.1 to 10,000 ppm (wt) |
| Allicin | 0.1 to 10,000 ppm (wt) |
| Cinnamaldehyde | 0.1 to 10,000 ppm (wt) |
| Oleuropein | 0.1 to 10,000 ppm (wt) |

### Example Formula 3

| Component | Level |
|---|---|
| mono- and di-glycerides of lauric (C12:0) | 0.01 to 50% (wt) |
| acid (monolaurin) | |
| Curcuminoids | 0.1 to 500000 ppm (wt) |
| Thymol | 0.1 to 250000 ppm (wt) |
| Carvacrol | 0.1 to 250000 ppm (wt) |
| Thymoquinone | 0.1 to 100000 ppm (wt) |

In general, compositions of the present technology, and products comprising such compositions, may be made by admixture of the components. In some aspects, aqueous products may be formulated in processes where the monoglycerides are blended into an aqueous mixture of the botanical components, optionally with heating. Optional emulsifiers may be used, as noted above.

In various embodiments, processes for making compositions comprise creating an admixture of components under heat (e.g., a temperature greater than about 38° C., such as between about 49° C. and 60° C.). The admixture is formed by mixing monoglycerides in order of carbon chain length (i.e., starting with monoglyceride having the shortest carbon chain (e.g., monopropionine) first, and ending with addition of the monoglyceride having the longest carbon chain length (e.g., monolaurin). The essential oils and curcuminoid are then added with surfactant and emulsifying agents. The resulting mixture is then mixed using adequate shear force and energy to create a stable emulsion. Water is then gradually added to the emulsion to create an oil in water emulsion that may be a microemulsion, or a nanoemulsion depending on the final application of the composition, or subsequent further dilutions for other applications.

As discussed above, the compositions of the present technology may be in any dosage form suitable for administration to humans or other animals. In various embodiments, the dosage forms are suitable for oral administration, parenteral, buccal, topical, nasal, or pulmonary administration.

For example, compositions may be delivered to livestock through animal feed, oral supplementation by some device (drench, paste gun, or scoop), topical skin cream, or through drinking water. In various embodiments, feed supplements of the present technology comprise a carrier to facilitate mixing with a base feed composition and effective delivery to the livestock animal. Such carriers include those known in the art. The feed supplements may optionally comprise mineral oil and/or vegetable or animal derived oils for fat soluble ingredients, as a feed additive and to reduce dustiness.

The present technology also provides animal food compositions comprising a bulk feed material and composition of the present technology. Bulk food material useful in this technology includes materials known in the art for feeding to livestock. For example, the bulk food material may contain grains, such as corn, processed soybeans, sorghum, wheat, rye, triticale, oats, barley and combinations thereof. Feed compositions may comprise a bulk feed material and a composition of the present technology, at levels of from about 0.5 to about 15, from about 1 to about 10, or from about 2 to about 7 pounds of the feed supplement per ton of feed composition.

In some embodiments, the compositions may be administered to humans orally as a nutritional supplement or food additive (e.g. nutritional shake, capsule form, powder form, paste), through a ready-made drink supplement, a powder blended into ready-made meals, or other forms of food delivery such as bread, pasta or other preparations. In some embodiments, compositions of the present technology may be liquid, such a “nutritional” supplement drink. In various embodiments, compositions may be formulated to coat the gastrointestinal tract (e.g., mouth and throat) of the subject ingesting the composition.

Alternatively, compositions of the present technology, or components thereof (e.g., one or more of monoglycerides, a curcuminoid, thymoquinone, thymol, and carvacrol) may be administered topically to tissues of the respiratory tract. In various embodiments, compositions for respiratory delivery comprise a composition or component of the present technology with a pharmaceutically-acceptable carrier, such as saline. The present technology provides products comprising such compositions and a respiratory delivery device, such as an aerosol or spray dispenser. For example, compositions may be formulated for inhalation, for delivery to throat and lung tissue, using a suitable device for pulmonary administration, including devices known in the art. In other embodiments, compositions may be administered by intranasal administration using suitable devices for intranasal administration, including devices known in the art. Advantageously, in various embodiments, administration of compositions coats the mucosa to provide a protective barrier or local delivery of the compositions.

The present technology also provides unit dosage form products, comprising a composition of the present technology in a safe and effective amount suitable for administration to a human or animal subject in a single dose. For example, such unit dose form products for oral administration to a human may comprise from about 50 mg to about 8000 mg, or from about 100 mg to about 4000 mg, or from about 500 mg to about 1000 mg, of a composition of the present technology.

### Methods

In various aspects, the present technology provides methods for mediating a nutritional or physiological status of a human or other animal, comprising administering to the subject a composition of the present technology. In various aspects, such methods are for the prevention, treatment or providing nutritional or other benefits in humans or other animals, such as when the subject has, or is at risk of having, a viral-mediated disorder. For example, methods may provide one or more benefits, including one or more of systemic or topical mucosal, neurological, pulmonary, and cardiovascular protection against viruses, treatment of viral infections, nutritional support, and support of body function (e.g., innate immune function or specific immune response).

Such methods may be nutritional, nutraceutical or pharmaceutical. It will be appreciated, though, that description or characterization of a composition or method in this application, or the prosecution of this application (including applications and issued patents claiming priority to this application), does not, in and of itself, constitute promotion of any given composition of the present technology as a pharmaceutical, even though such compositions may have utility in the treatment or prevention of a viral-mediated disorder or other physiological or pharmacological benefit.

In particular, the present technology provides methods for providing a benefit (e.g., nutritional, nutraceutical or pharmaceutical, as further discussed herein) to a human or other animal subject comprising administering to the subject a safe and effective amount of a composition of the present technology. The “safe and effective” amount of the composition is an amount that is sufficient to provide the intended benefit in the subject, without undue adverse side effects (such as toxicity, irritation, or allergic response), commensurate with a reasonable benefit/risk ratio when used in the manner of this technology. The specific safe and effective amount of the adrenergic compound will, obviously, vary with such factors as the particular benefit to be provided (e.g., the condition to be prevented or treated), the physical condition of the subject, the nature of concurrent therapy (if any), the specific composition used, the specific route of administration and dosage form, the carrier employed, and the desired dosage regimen. Exemplary benefits provided by methods include any one or more of the benefits described herein.

In general, methods of the present technology may comprise administering a total dosage of from about 5 mg per kg body weight to about 100 mg per kg body weight, or from about 15 mg per kg body weight to about 40 mg per kg body weight, or from about 20 mg per kg body weight to about 30 mg per kg body weight of a composition of the present technology to a human subject, in from 1 to 6, or 2 to 4 doses per day. The present technology provides administration regiments, wherein compositions of the present technology are administered each day for from 1 day to about 6 months, or from 3 days to about 1 month, or from about 5 days to about 4 weeks, or from about 1 week to about 2 weeks. In some embodiments, such as for providing nutritional benefits or preventing a viral disorder, compositions are administered daily for an indefinite period of time.

Viral disorders among those prevented, treated or ameliorated by the methods of this technology include those caused by helical, icosahedral, prolate, envelope, complex or giant viruses. In particular, viruses includes DNA viruses, RNA viruses and reverse transcribing viruses. Viruses include those from the families Adenoviridae, Anelloviridae, Arenaviridae, Astroviridae, Bornaviridae, Bunyaviridae, Caliciviridae, Coronaviridae, Filoviridae, Flaviviridae, Hepadnaviridae, Hepeviridae, Herpesviridae, Orthomyxoviridae, Papillomaviridae, Paramyxoviridae, Parvoviridae, Picobirnaviridae, Picobirna, Picomaviridae, Pneumoviridae, Polymaviridae, Poxiviridae, Reoviridae, Retroviridae, Rhabdoviridae, Togaviridae, and Delta.

Viral-mediated disorders include disorders in livestock and other animals associated with viruses, including Porcine Epidemic Diarhea Virus (Coronaviridae PEDv), Transmissible Gastroenteritis (Coronaviridae TGE), Porcine Respiratory Reproductive Syndrome (Togaviridae PRRS), African Swine Fever Virus (Asfarviridae ASFv) Foot-and-Mouth Virus (Picornaviridae FMDV), Seneca Valley Virus (Picornaviradae SVA), Influenza A (Orthomyxoviridae), Avian Influenza H5N1, and Rotavirus A, Rotavirus B, Rotavirus C, Rotavirus D (Reoviridae). In various aspects, methods of the present technology improve livestock welfare, performance, and survivability, and may reduce the incidence of death.

Viral-mediated disorders include disorders in humans associated with viruses, including Adenovirus, Chikungunya Virus, Cytomegalovirus, Dengue virus, Ebola Virus, Enterovirus, Hantavirus, HIV, Hepatitis B, Hepatitis C, Human Metapneumovirus (HMPV), Human Parainfluenza Viruses (HPIVs), Influenza A (e.g., H1N1), Influenza B, Marburg virus, Measles (Rubeola) Virus, MERS-CoV, Oncoviruses, Parvovirus B19, Rabies, Respiratory Syncytial Virus (RSV), Rotavirus, Rubella virus, SARS-CoV, SARS-CoV-2 (COVID-19), West Nile Virus, Varicella-Zoster Virus, Zika Virus. In various embodiments, compositions and methods of the present technology provide benefits in individuals having, or at risk of having a viral-mediated disorder associated with viruses of the family Coronaviridae (e.g., MERS-CoV, SARS-CoV, SARS-CoV-2), and viruses of the family Orthomyxoviridae (e.g., Influenza A, Influenza B).

Viruses are transmitted in various ways, but often are swallowed, inhaled, spread by bites from insects, spread through sexual activity, and from blood transfusion. The oral route is the most common and, in various aspects, the present technology acts to reduce the probability of infection through systemic protection of mucosal tissues and points of entry for viruses.

Viral disease challenges lead to a vast range of physiological symptoms including runny nose, sore throat, cough, congestion, body aches and headache, sneezing, fever, feeling unwell, difficulty breathing, abdominal pains, vomiting, diarrhea, rash or bleeding. As noted above, viral disorders may include hypercytokinemia (“cytokine storm”) or cytokine release syndrome, which may lead to severe fever, or severe bleeding which may lead to death. In various embodiments, the compositions and methods of the technology support and adjust the hosts immune response to prevent hypercytokinemia and reduce the probability of severe disease or death by immune system modulation and homeostasis.

Without limiting the scope or mechanism of the present technology, it is believed that compositions and methods of the present technology, in some embodiments, mediate viral-disorders by denaturing viral envelopes and structure, interfering with RNA or DNA replication, or otherwise reduce the growth rate of virus in mucosal layers, in the blood, gastrointestinal tract and other parts of the subject to whom the compositions are administered. In various aspects, compositions may allow the immune system of the subject to overcome the viral challenge, and, at the same time, through the joint action of alpha monolaurin, curcuminoids and thymoquinone work to regulate the host's immune system to minimize the negative physiological consequences of infection such as cytokine cascades and fever, and support development of acquired immunity to naturally fight against the viral infection. In various aspects, through the dual action of acting on the virus, and acting on the host, the technology reduces and prevents the effects of viral disease challenges

In various embodiments, these actions may be provided by mono-glycerides in combination with the thymol and carvacrol for penetrating the viral structure, and the synergistic effects of curcumin and thymoquinone on RNA replication. Accordingly, in various aspects, the present technology provides a select combination of compounds (curcumin, thymoquinone and monolaurin) to regulate the immune system to support a direct immune response against pathogens, yet reducing the extent to which inflammation and over-expression of immunity can cause issues.

In some embodiments, without limiting the scope or mechanism of the present technology, the combination of short, medium and long chain monoglycerides creates a systemic level of protection through acting in the oral mucosa, esophagus, and full gastrointestinal tract, as well as by being both hydrophilic and in the blood and lymphatic system.

Further, in some aspects, without limiting the scope or mechanism of the present technology, the combination of curcuminoids, thymol, carvacrol and thymoquinone interfere with growth and establishment of viruses, in some cases preventing their attachment or incorporation into mucosal membranes where they begin to replicate, and modulate the immune system through supporting the unique resource needs of the immune system, and maintain homeostatic balance of the immune system to prevent over-reaction of the immune system that often can lead to death of its host.

Accordingly, in various aspects, the present technology provides methods for preventing, treating or providing nutritional benefits relating to viral-mediated disorders in humans or other animal subjects, comprising administering to the subject a composition of the present technology. In various aspects, such methods include improving the nutritional or immunological status of a human or other animal. In some aspects, the present technology provides nutritional compositions and methods for improving or supporting the nutritional status or immune function of humans or other animal subjects that may be or have been exposed to viral pathogens, or that have or may exhibit symptoms of viral infection. Methods may also prevent, reduce the occurrence of, or the severity of symptoms associated with viral pathogens. In various aspects, nutritional benefits include maintaining, strengthening or improving immune function, maintaining, strengthening or improving immune status, maintaining, strengthening or improving immune health, maintaining, strengthening or improving innate immune function, maintaining, strengthening or improving respiratory function, reducing the duration of symptoms associated with respiratory disease, reducing the severity of symptoms associated with respiratory disease, and combinations thereof.

In some aspects, compositions and methods are administered to subjects having failing, naive, or compromised immune systems. Accordingly, in some embodiments, methods of the present technology mediate expression or production of cytokines associated with viral infections, such as IL-6 and IL-8, but may influence other cytokines and chemokines.

In some aspects, the present technology provides compositions for the prevention or treatment of viral-mediated disorders, such as for the prevention or treatment of viral infections. For example, compositions and methods are safe and effective for treatment, prevention or alleviating the symptoms of diseases and other disorders (e.g., hypercytokinemia) caused by viral pathogens.

In some embodiments, such as those relating to the prevention of viral disorders, the subject may be at risk of infection, due to microbes that are present in the environment, e.g., in an “at risk” group of human subjects in a health care setting, nursing home, school or other environments where transmission of viruses through contact or air transmission is possible. For livestock, an animal may be a member of a herd or other group of livestock in which the subject is a member. Thus, methods of prevention reduce the likelihood of disease in the subject. It should be recognized that, as to any individual subject not having an infection at the time a composition of the present technology is administered, it may not be definitively known whether the subsequent lack of infection was, in fact, due to the administration of the composition or for some other reasons (i.e., an actual lack of microbial exposure). Rather, the effectiveness of the method of preventing infection may be seen only in the reduction of the incidence of infections in a population of subjects. Thus, as used herein, “preventing” refers to reducing the rate or probability of infections associated with pathogens, in a livestock subject at risk of infection.

Accordingly, in various embodiments, compositions of the present technology are administered to a subject continuously as a preventative measure for viral infection or symptoms, and to reduce symptoms and physiological changes such as the cytokine cascade that leads to illness and reducing fever, and reducing damage to tissue caused either directly by the virus, or by the host's own immune system in fighting with the virus. In some aspects methods alleviate morbidity (and potential death) in people infected with SARS-CoV-2 (COVID-19), or other emerging forms of viruses that negatively affect specific parts of the population such as the very young, the very old or people with other health challenges. For example, such methods may alleviate the large fever, and cytokine cascade caused by SARS-CoV-2 (COVID-19), and thereby support people through the infection, to recovery. In some embodiments, methods alleviate and protect against illness for subjects in “at risk” populations that are exposed continuously to viruses, such as SARS-CoV-2 either working in health care, emergency services, or in the retail or travel industry.

As discussed above, methods may alleviate the level of disease (and potential death) in people infected with SARS-CoV-2 (COVID-19), or other emerging forms of viruses that negatively affect specific parts of the population such as the very young, the very old or people with other health challenges. Such methods may alleviate the large fever, and cytokine cascade caused by SARS-CoV-2 (COVID-19), and thereby support people through the infection, to recovery. In some embodiments, methods alleviate and protect against illness for people that are exposed continuously to viruses such as SARS-CoV-2, working (for example) in health care, emergency services, or in the retail or travel industry.

In some embodiments, the compositions and methods of the present technology may be useful in preventing, treating or ameliorating the symptoms of COVID-19. In one aspect, such methods may reduce how many individuals (e.g., humans) are infected from exposure to an infected person (reduced transmission rate). Methods may reduce excretion of virus from those infected (reduced environmental contamination), for example in non-symptomatic hosts where fecal-to-oral transmission may be source of virus. In other aspects, methods may provide beneficial support in overcoming an existing challenge with SARS-COV-2 in challenged patients. In some embodiments, the compositions and methods of the present technology may mediate extreme immune responses (e.g., “cytokine storms”) associated with COVID-19 and other viral infections.

In various aspects, the compositions and methods of the present technology selectively support the innate and adaptive immune system to respond adequately, when exposed to viruses, and thereby reduce the risk of severe illness or death. In various aspects, without limiting the scope or mechanism of the present technology, such compositions and methods support the innate immune system by creating a “systemic entry barrier” to the viruses by integrating into mucosal layers or other absorptive surfaces where viruses take hold such as nostrils, mouth, throat, stomach or cardiovascular system. Compositions may be administered prior to exposure to a virus for greatest effectiveness but can also be administered after exposure to a virus to support the homeostasis of the body. The present technology may support the immune system to also mount adequate acquired immunity so that once the virus has been recognized, and the body has acquired immunity to that specific virus that through the key support provided by the technology, the immune system will be able to overcome the challenge. In addition, the present compositions may have direct influence on the proliferation of viruses through either interrupting their replication, or their structure or by some other means. Without limiting the mechanism or scope of the present technology, the compositions may achieve this through the means of a nutritional supplement that is safe to consume, with minimal toxicity.

The present technology also provides methods of preventing, treating or providing nutritional or other benefits relating to viral-mediated disorders comprising administering a composition of the present technology and a viral treatment agent. Such viral treatment agents include agents that prevent or treat viral disorders or associated symptoms. For example, such agents for the treatment of COVID-19 include anti-virals (e.g., remdesivir, R-D-N4-hydroxycytidine, favipiravir, and merimepodib), dexamethasone, anti-inflammatory drugs, immune-based therapies (e.g., convalescent plasma), chloroquine, and azithromycin. In some methods, the antiviral agent has a mechanism of action that interferes with a critical viral enzyme, such as an RNA polymerase. In some embodiments, methods include administration of a composition of the present technology with remdesivir for the treatment of COVID-19 or other viral diseases. Without limiting the scope or mechanism of the present technology, the compositions of the present technology may limit or prevent entry of virus into cells (e.g., in the gastroinstinal tract) while remdesivir acts to directly reduce viral replication (i.e., by interfering with RNA-dependent RNA polymerase).

The present technology also provides methods of immunizing a human or other animal subject comprising administering a composition of the present technology and an immunization agent. Without limiting the scope or mechanism of the present technology, in various embodiments the compositions of the present technology may serve as a vaccination adjuvant, enhancing the immunologic response of the vaccination agent in the subject. Vaccination agents include any agent which provides active acquired immunity to a virus in a human or other animal subject leading to a stronger and more effective immune response, allowing lower peak viral loads, less damage, and faster rates of recovery. Such agents may comprise weakened or killed forms of the virus or a component of the virus (e.g., a surface protein or nucleic acid). For example, the present technology provides methods for immunizing a subject for COVID-19 or other SARS-associated virus by administering a vaccination agent and a composition of the present technology. Such methods may comprise administration of the vaccination agent as a single injection, or two or more injections over a period of time, together with administration of a composition of the present technology proximate to the time(s) of the injection(s), e.g., from 1 day to 90 days before and/or after the injection(s). Without limiting the scope or mechanism of the present technology, such methods may provide protection of the gastrointestinal tract (e.g., small intestine) of the subject by ingestion of a composition of the present technology in combination with prevention of respiratory or other systemic effects by administration of the immunization agent. In various embodiments, the compositions of the present technology may increase the level or effectiveness of antibodies generated by the immunization agent.

As noted above, the compositions and methods of the present technology may be used with non-human animals, such as companion animals and livestock. Companion animals include dogs and cats. Livestock includes any animal (including birds) kept for commercial purposes, such as domesticated animals that are raised for producing commodities such as food (e.g., milk and meat), animal products (e.g., fiber), or working in an agricultural or other commercial activity. Examples include pigs, cows, horses, sheep, chicken and goats. In some embodiments, livestock are monogastric. In a preferred embodiment, a livestock animal is a pig.

Compositions and methods may also be used with humans, including male and female infants, children, and adults (including adults over the age of 65, or over the age of 70, or over the age of 80, or over the age of 90). In some aspects, compositions and methods may be used with humans at risk of death, morbidity or complications associated with viral infections, such as subject having comorbidity or “pre-existing” conditions that increase the duration, severity (or both) of a viral-mediated disorder. Such conditions include, for example, diabetes, heart disease (e.g., congestive heart failure), obesity (e.g., having a BMI (body mass index)>30), chronic lung disease (e.g., chronic obstructive pulmonary disease, emphysema, chronic bronchitis, cystic fibrosis, idiopathic pulmonary fibrosis), cancer, sickle cell disease, and chronic kidney disease. Accordingly, the present technology provides methods of administering nutritional compositions to subjects at risk of a viral-mediated disorder wherein the subject has a comorbidity.

In various embodiments, methods provide benefits related to neurological or behavioral benefits. In various embodiments, compositions of the present technology provide clarity of mind in subjects to whom they are administered, such as continued focus and maintained focus levels over extended periods of time, e.g., throughout a day. Compositions may afford increased overall energy levels. For example, administration of the composition in the morning may impact energy and focus levels later in the afternoon. In various aspects, this may afford benefits for subjects working long shifts, night shifts, driving vehicles for prolonged periods, or people working with tasks such as computer based, or screen based work, that requires significant focus and concentration to be maintained. Accordingly, in various embodiments, the present technology provides methods: for maintaining, improving or strengthening cognitive function; maintaining, strengthening or improving mental focus; maintaining, strengthening or improving mental acuity; maintaining, strengthening or improving memory; maintaining, strengthening or improving functional capacity; maintaining, strengthening or improving emotional well-being; maintaining, strengthening or improving endurance performance; maintaining, strengthening or improving endurance capacity; maintaining, strengthening or improving alertness; maintaining, strengthening or improving concentration; providing energy; and reducing fatigue.

### Non-Limiting Listing of Exemplary Embodiments

The present technology includes the following exemplary embodiments.
A1. A composition for administration to a human or other animal subject, comprising
   a monoglyceride;
   a curcuminoid;
   thymoquinone;
   thymol;
   carvacrol; and
   p-cymene.
A2. The composition of Embodiment A1, wherein the monoglyceride comprises an alpha-monoglyceride.
A3. The composition of either Embodiment A1 or A2, wherein the monoglyceride comprises an acid selected from the group consisting of formic acid, acetic acid, propionic acid, butyric acid, valeric acid, caproic acid, caprylic acid, capric acid, lauric acid, myristic acid, pentadecanoic acid and hexadecenoic acid.
A4. The composition of one of Embodiments A1-A3, wherein the monoglyceride comprises an acid selected from the group consisting of propionic acid, butyric acid, caproic acid, and lauric acid.
A5. The composition of any one of Embodiments A1-A4, wherein the monoglyceride comprises monolaurin, or is monolaurin.
A6. The composition of any one of Embodiments A1-A5, wherein the monoglyceride comprises a mixture of alpha-monoglycerides.
A7. The composition of any one of Embodiments A1-A6, wherein the monoglyceride is present at a level of from about 30% to about 70%, or from about 40% to about 60%, or from about 45% to about 55%.
A8. The composition of any one of Embodiments A1-A7, comprising a turmeric extract, wherein the turmeric extract comprises the curcuminoid.
A9. The composition of any one of Embodiments A1-A8, wherein the curcuminoid is a component of an extract, the extract being present in the composition at a level of from about 0.1% to about 5%, or from about 0.2% to about 2%, or from about 0.5% to about 1%.
A10. The composition of any one of Embodiments A1-A9, wherein the thymoquinone is present at a level of from about 0.01% about 0.2%.
A11. The composition of any one of Embodiments A1-A10, wherein the thymol is present at a level of from about 0.01% about 0.2%.
A12. The composition of any one of Embodiments A1-A11, wherein the carvacrol is present at a level of from about 0.01% about 0.2%.
A13. The composition of any one of Embodiments A1-A12, comprising a Monarda extract, wherein the Monarda extract comprises the thymoquinone, the thymol, and the carvacrol.
A14. The composition of Embodiment A13, wherein the Monarda extract is present at a level of from about 0.1% to about 5%, or from about 0.2% to about 2%, or from about 0.5% to about 1%.
A15. A composition of any one of Embodiments A1-A14, further comprising a carrier suitable for administration to a human or animal subject.
A16. A composition for the prevention or treatment of a viral-mediated disorder in a human or animal subject, comprising a pharmaceutically safe and effective amount of a composition of any one of Embodiments A1-A15.
A17. The composition of Embodiment A16, for oral, parenteral, buccal, nasal or pulmonary administration.
A18. A composition for nutritional support of a human or animal subject comprising a nutritionally safe and effective amount of a composition of any one of Embodiments A1-A15.
A19. The composition of Embodiment A18 for oral administration further comprising a flavoring agent.
A20. The composition of any one of Embodiments A15-A19, for administration to a non-human animal.
A21. An animal food composition comprising the composition of Embodiment A20.
A22. The composition of any one of Embodiments A15-A19, for administration to a human.
A23. The composition of Embodiment A22, in the form of a nutritional supplement or food additive.
A24. A food composition comprising the composition of either Embodiment A22 or Embodiment A23.
B1. A composition for administration to a human or other animal subject, comprising
   a monoglyceride;
   a curcuminoid; and
   a Monarda extract comprising thymoquinone, thymol, carvacrol, and p-cymene.
B2. The composition of Embodiment B1, wherein the monoglyceride comprises an alpha-monoglyceride.
B3. The composition of either Embodiment B1 or B2, wherein the monoglyceride comprises an acid selected from the group consisting of formic acid, acetic acid, propionic acid, butyric acid, valeric acid, caproic acid, caprylic acid, capric acid, lauric acid, myristic acid, pentadecanoic acid and hexadecenoic acid.
B4. The composition of one of Embodiments B1-B3, wherein the monoglyceride comprises an acid selected from the group consisting of propionic acid, butyric acid, caproic acid, and lauric acid.
B5. The composition of any one of Embodiments B1-B4, wherein the monoglyceride comprises monolaurin, or is monolaurin.
B6. The composition of any one of Embodiments B1-B5, wherein the monoglyceride comprises a mixture of alpha-monoglycerides.
B7. The composition of any one of Embodiments B1-B6, wherein the monoglyceride is present at a level of from about 30% to about 70%, or from about 40% to about 60%, or from about 45% to about 55%.
B8. The composition of any one of Embodiments B1-B7, comprising a turmeric extract, wherein the turmeric extract comprises the curcuminoid.
B9. The composition of any one of Embodiments B1-B8, wherein the curcuminoid is a component of an extract, the extract being present in the composition at a level of from about 0.1% to about 5%, or from about 0.2% to about 2%, or from about 0.5% to about 1%.
B10. The composition of any one of Embodiments B1-B9, wherein the thymoquinone is present at a level of from about 0.01% about 0.2%.
B11. The composition of any one of Embodiments B1-B10, wherein the thymol is present at a level of from about 0.01% about 0.2%.
B12. The composition of any one of Embodiments B1-B11, wherein the carvacrol is present at a level of from about 0.01% about 0.2%.
B13. The composition of any one of Embodiments B1-B12, wherein the Monarda extract is present at a level of from about 0.1% to about 5%, or from about 0.2% to about 2%, or from about 0.5% to about 1%.
B14. A composition of any one of Embodiments B1-B13, further comprising a carrier suitable for administration to a human or animal subject.
B15. A composition for the prevention or treatment of a viral-mediated disorder in a human or animal subject, comprising a pharmaceutically safe and effective amount of a composition of any one of Embodiments B1-B14.
B16. The composition of Embodiment B15, for oral, parenteral, buccal, nasal or pulmonary administration.
B17. A composition for nutritional support of a human or animal subject comprising a nutritionally safe and effective amount of a composition of any one of Embodiments B1-B14.
B18. The composition of Embodiment B15 for oral administration further comprising a flavoring agent.
B19. The composition of any one of Embodiments B14-B18, for administration to a non-human animal.
B20. An animal food composition comprising the composition of any one of Embodiments B14-B19.
B21. The composition of any one of Embodiments B14-B18, for administration to a human.
B22. The composition of Embodiment B21, in the form of a nutritional supplement or food additive.
B23. A food composition comprising the composition of either Embodiment B21 or Embodiment B22.
C1. A composition for administration to a human or other animal subject, comprising
   monolaurin;
   a curcuminoid; and
   thymoquinone.
C2. The composition of Embodiment C1, wherein the monolaurin is present at a level of from about 30% to about 70%, or from about 40% to about 60%, or from about 45% to about 55%.
C3. The composition of either Embodiment C1 or Embodiment C2, comprising a turmeric extract, wherein the turmeric extract comprises the curcuminoid.
C4. The composition of any one of Embodiments C1-C3, wherein the curcuminoid is present at a level of from about 0.1% to about 5%, or from about 0.2% to about 2%, or from about 0.5% to about 1%.
C5. The composition of any one of Embodiments C1-C4, wherein the thymoquinone is present at a level of from about 0.1 to about 5%, or from about 0.01% to about 0.2%.
C5. The composition of any one of Embodiments C1-C5, comprising a Monarda extract, wherein the Monarda extract comprises the thymoquinone, the thymol, and the carvacrol.
C6. The composition of Embodiment C5, wherein the Monarda extract is present at a level of from about 0.1% to about 5%, or from about 0.2% to about 2%, or from about 0.5% to about 1%.
C7. A composition of any one of Embodiments C1-C6, further comprising a carrier suitable for administration to a human or animal subject.
C8. A composition for the prevention or treatment of a viral-mediated disorder in a human or animal subject, comprising a pharmaceutically safe and effective amount of a composition of Embodiment C1-C7.
C9. The composition of Embodiment C8, for oral, parenteral, buccal, nasal or pulmonary administration.
C10. A composition for nutritional support of a human or animal subject comprising a nutritionally safe and effective amount of a composition of any one of Embodiments C1-C9.
C11. The composition of Embodiment C10 for oral administration, further comprising a flavoring agent.
C12. The composition of either of Embodiment C10 or Embodiment C11, for administration to a non-human animal.
C13. A animal food composition comprising the composition of any one of Embodiments C10-C12.
C14. The composition of either of Embodiment C10 or Embodiment C11, for administration to a human.
C15. The composition of Embodiment C14, in the form of a nutritional supplement or food additive.
C16. A food composition comprising the composition of either Embodiment C14 or Embodiment C15.
D1. A method for mediating a nutritional or physiological status of a human or animal subject comprising administering to the subject a composition comprising
   a monoglyceride;
   a curcuminoid;
   thymoquinone;
   thymol;
   carvacrol; and
   p-cymene.
D2. The method of Embodiment D1, wherein the monoglyceride comprises an alpha-monoglyceride.
D3. The method of either Embodiment D1 or Embodiment D2, wherein the monoglyceride comprises a carboxylic acid selected from the group consisting of formic acid, acetic acid, propionic acid, butyric acid, valeric acid, caproic acid, caprylic acid, capric acid, lauric acid, myristic acid, pentadecanoic acid and hexadecenoic acid.
D4. The method of one of Embodiments D1-D3, wherein the monoglyceride comprises a carboxylic acid selected from the group consisting of propionic acid, butyric acid, caproic acid, and lauric acid.
D5. The method of any one of Embodiments D1-D4, wherein the monoglyceride comprises monolaurin, or is monolaurin
D6. The method of any one of Embodiments D1-D5, wherein the monoglyceride comprises a mixture of alpha-monoglycerides.
D7. The method of any one of Embodiments D1-D6, wherein the monoglyceride is present at a level of from about 30% to about 70%, or from about 40% to about 60%, or from about 45% to about 55%.
D8. The method of any one of Embodiments D1-D7, comprising a turmeric extract, wherein the turmeric extract comprises the curcuminoid.
D9. The method of any one of Embodiments D1-D8, wherein the curcuminoid is a component of an extract, the extract being present in the composition at a level of from about 0.1% to about 5%, or from about 0.2% to about 2%, or from about 0.5% to about 1%.
D10. The method of any one of Embodiments D1-D9, wherein the thymoquinone is present at a level of from about 0.01% about 0.2%.
D11. The method of any one of Embodiments D1-D10, wherein the thymol is present at a level of from about 0.01% about 0.2%.
D12. The method of any one of Embodiments D1-D11, wherein the carvacrol is present at a level of from about 0.01% about 0.2%.
D13. The method of any one of Embodiments D1-D12, comprising a Monarda extract, wherein the Monarda extract comprises the thymoquinone, the thymol, and the carvacrol.
D14. The method of Embodiment D13, wherein the Monarda extract is present at a level of from about 0.1% to about 5%, or from about 0.2% to about 2%, or from about 0.5% to about 1%.
D15. A method of any one of Embodiments D1-D14, further comprising a carrier suitable for administration to a human or animal subject.
D16. A method for maintaining or improving the nutritional status of a human or animal subject according to the method of any one of Embodiments D1-D15.
D17. The method of any one of Embodiments D1-D16, wherein the subject has or is at risk of having a viral-mediated disorder.
D18. The method of Embodiment D17 wherein the viral-mediated disorder is associated with viruses of the family Coronaviridae (e.g., MERS-CoV, SARS-CoV, SARS-CoV-2), or viruses of the family Orthomyxoviridae (e.g., Influenza A, Influenza B).
D19. The method of Embodiment D17 wherein the viral-mediated disorder is associated with Porcine Epidemic Diarrhea Virus, Transmissible Gastroenteritis, Porcine Respiratory Reproductive Syndrome, African Swine Fever Virus, Foot-and-Mouth Virus, Seneca Valley Virus, Influenza A, Avian Influenza H5N1, Rotavirus A, Rotavirus B, Rotavirus C, or Rotavirus D.
D20. The method of any of Embodiments D17-D19, wherein the subject has pneumonia.
D21. The method of any of Embodiments D17-D20, wherein the subject has severe acute respiratory syndrome.
D22. A method for preventing or treating a viral-mediated disorder in a human or other animal according to the method of any one of Embodiments D1-D15.
D23. The method of Embodiment D22 for preventing or treating a viral-mediated disorder in humans associated with viruses of the family Coronaviridae (e.g., MERS-CoV, SARS-CoV, SARS-CoV-2), or viruses of the family Orthomyxoviridae (e.g., Influenza A, Influenza B).
D24. The method of Embodiment D22 for preventing or treating a viral-mediated disorder in livestock associated with Porcine Epidemic Diarrhea Virus, Transmissible Gastroenteritis, Porcine Respiratory Reproductive Syndrome, African Swine Fever Virus, Foot-and-Mouth Virus, Seneca Valley Virus, Influenza A, Avian Influenza H5N1, Rotavirus A, Rotavirus B, Rotavirus C, or Rotavirus D.
D25. A method for providing a nutritional benefit in a human or animal subject according to the method of any one of Embodiments D1-D15.
D26. The method of Embodiment D25, wherein the nutritional benefit is selected from the group consisting of maintaining, strengthening or improving immune function, maintaining, strengthening or improving immune status, maintaining, strengthening or improving immune health, maintaining, strengthening or improving innate immune function, maintaining, strengthening or improving respiratory function, reducing the duration of symptoms associated with respiratory disease, reducing the severity of symptoms associated with respiratory disease, and combinations thereof.
D26. The method of Embodiment D25, wherein the nutritional benefit is cognitive.
D27. The method of Embodiment D26, wherein the benefit selected from the group consisting of maintaining, improving or strengthening cognitive function; maintaining, strengthening or improving mental focus; maintaining, strengthening or improving mental acuity; maintaining, strengthening or improving memory; maintaining, strengthening or improving functional capacity; maintaining, strengthening or improving emotional well-being; maintaining, strengthening or improving endurance performance; maintaining, strengthening or improving endurance capacity; maintaining, strengthening or improving alertness; maintaining, strengthening or improving concentration; providing energy; reducing fatigue; and combinations thereof.
D28. A method for maintaining or improving the cognitive function of a human or animal subject, comprising administering to the subject a composition according to any one of Embodiments A1-C16.
D29. The method of Embodiment D28, wherein the benefit selected from the group consisting of maintaining, improving or strengthening cognitive function; maintaining, strengthening or improving mental focus; maintaining, strengthening or improving mental acuity; maintaining, strengthening or improving memory; maintaining, strengthening or improving functional capacity; maintaining, strengthening or improving emotional well-being; maintaining, strengthening or improving endurance performance; maintaining, strengthening or improving endurance capacity; maintaining, strengthening or improving alertness; maintaining, strengthening or improving concentration; providing energy; reducing fatigue; and combinations thereof.
E1. A method for maintain or improving a nutritional status of a human or animal subject having or at risk of having a viral-mediated disorder, comprising administering to the subject a composition comprising two or more of the following components:
   a monoglyceride;
   a curcuminoid;
   thymoquinone;
   thymol;
   carvacrol; and
   p-cymene.
E2. The method of Embodiment E1, wherein the composition comprises three or more of the components.
E3. The method of Embodiment E1, wherein the composition comprises four or more of the components.
E4. The method of any one of Embodiments E1-E3, wherein the monoglyceride comprises an alpha-monoglyceride.
E5. The method of any one of Embodiments E1-E4, wherein the monoglyceride comprises a fatty acid selected from the group consisting of formic acid, acetic acid, propionic acid, butyric acid, valeric acid, caproic acid, caprylic acid, capric acid, lauric acid, myristic acid, pentadecanoic acid and hexadecenoic acid.
E6. The method of one of Embodiments E1-E5, wherein the monoglyceride comprises a carboxylic acid selected from the group consisting of propionic acid, butyric acid, caproic acid, and lauric acid.
E7. The composition of any one of Embodiments E1-E6, wherein the monoglyceride comprises monolaurin, or is monolaurin
E8. The method of any one of Embodiments E1-E7, wherein the monoglyceride comprises a mixture of alpha-monoglycerides.
E9. The method of any one of Embodiments E1-E8, wherein the monoglyceride is present at a level of from about 30% to about 70%, or from about 40% to about 60%, or from about 45% to about 55%.
E10. The method of any one of Embodiments E1-E9, comprising a turmeric extract, wherein the turmeric extract comprises the curcuminoid.
E11. The method of any one of Embodiments E1-E10, wherein the curcuminoid is a component of an extract, the extract being present in the composition at a level of from about 0.1% to about 5%, or from about 0.2% to about 2%, or from about 0.5% to about 1%.
E12. The method of any one of Embodiments E1-E11, wherein the thymoquinone is present at a level of from about 0.01% about 0.2%.
E13. The method of any one of Embodiments E1-E12, wherein the thymol is present at a level of from about 0.01% about 0.2%.
E14. The method of any one of Embodiments E1-E13, wherein the carvacrol is present at a level of from about 0.01% about 0.2%.
E15. The method of any one of Embodiments E1-E14, comprising a Monarda extract, wherein the Monarda extract comprises the thymoquinone, the thymol, and the carvacrol.
E16. The method of Embodiment E15, wherein the Monarda extract is present at a level of from about 0.1% to about 5%, or from about 0.2% to about 2%, or from about 0.5% to about 1%.
E17. A method of any one of Embodiments E1-E16, further comprising a carrier suitable for administration to a human or animal subject.
E18. The method of any one of Embodiments E1-E17, wherein the viral-mediated disorder is associated with viruses of the family Coronaviridae (e.g., MERS-CoV, SARS-CoV, SARS-CoV-2), or viruses of the family Orthomyxoviridae (e.g., Influenza A, Influenza B).
E19. The method of any one of Embodiments E1-E17, wherein the viral-mediated disorder is associated with Porcine Epidemic Diarrhea Virus, Transmissible Gastroenteritis, Porcine Respiratory Reproductive Syndrome, African Swine Fever Virus, Foot-and-Mouth Virus, Seneca Valley Virus, Influenza A, Avian Influenza H5N1, Rotavirus A, Rotavirus B, Rotavirus C, or Rotavirus D.
E20. The method of any one of Embodiments E1-E19, wherein the subject has pneumonia.
E21. The method of any one of Embodiments E1-E20, wherein the subject has severe acute respiratory syndrome.
F1. A composition for the prevention or treatment of a viral-mediated disorder in a human or other animal, comprising two or more of the following components:
   a monoglyceride;
   a curcuminoid;
   thymoquinone;
   thymol;
   carvacrol; and
   p-cymene.
F2. The composition of Embodiment F1, wherein the composition comprises three or more of the components.
F3. The composition of Embodiment F1, wherein the composition comprises four or more of the components.
F4. The composition of any one of Embodiments F1-F3, wherein the monoglyceride comprises an alpha-monoglyceride.
F5. The composition of any one of Embodiments F1-F4, wherein the monoglyceride comprises a fatty acid selected from the group consisting of formic acid, acetic acid, propionic acid, butyric acid, valeric acid, caproic acid, caprylic acid, capric acid, lauric acid, myristic acid, pentadecanoic acid and hexadecenoic acid.
F6. The composition of one of Embodiments F1-F5, wherein the monoglyceride comprises a carboxylic acid selected from the group consisting of propionic acid, butyric acid, caproic acid, and lauric acid.
F7. The composition of any one of Embodiments F1-F6, wherein the monoglyceride comprises monolaurin, or is monolaurin
F8. The composition of any one of Embodiments F1-F7, wherein the monoglyceride comprises a mixture of alpha-monoglycerides.
F9. The composition of any one of Embodiments F1-F8, wherein the monoglyceride is present at a level of from about 30% to about 70%, or from about 40% to about 60%, or from about 45% to about 55%.
F10. The composition of any one of Embodiments F1-F9, comprising a turmeric extract, wherein the turmeric extract comprises the curcuminoid.
F11. The composition of any one of Embodiments F1-F10, wherein the curcuminoid is a component of an extract, the extract being present in the composition at a level of from about 0.1% to about 5%, or from about 0.2% to about 2%, or from about 0.5% to about 1%.
F12. The composition of any one of Embodiments F1-F11, wherein the thymoquinone is present at a level of from about 0.01% about 0.2%.
F13. The composition of any one of Embodiments F1-F12, wherein the thymol is present at a level of from about 0.01% about 0.2%.
F14. The composition of any one of Embodiments F1-F13, wherein the carvacrol is present at a level of from about 0.01% about 0.2%.
F15. The composition of any one of Embodiments F1-F14, comprising a Monarda extract, wherein the Monarda extract comprises the thymoquinone, the thymol, and the carvacrol.
F16. The composition of Embodiment F15, wherein the Monarda extract is present at a level of from about 0.1% to about 5%, or from about 0.2% to about 2%, or from about 0.5% to about 1%.
F17. The composition of any one of Embodiments F1-F16, further comprising a carrier suitable for administration to a human or animal subject.
F18. The composition of Embodiment F17, wherein the carrier is operable for oral, nasal, or pulmonary administration.
G1. A method for the prevention or treatment of a viral-mediated disorder in a human or other animal subject, comprising administering to the subject a composition comprising two or more of the following components:
   a monoglyceride;
   a curcuminoid;
   thymoquinone;
   thymol;
   carvacrol; and
   p-cymene.
G2. The composition of Embodiment G1, wherein the composition comprises three or more of the components.
G3. The composition of Embodiment G1, wherein the composition comprises four or more of the components.
G4. The composition of any one of Embodiments G1-G3, wherein the monoglyceride comprises an alpha-monoglyceride.
G5. The composition of any one of Embodiments G1-G4, wherein the monoglyceride comprises a fatty acid selected from the group consisting of formic acid, acetic acid, propionic acid, butyric acid, valeric acid, caproic acid, caprylic acid, capric acid, lauric acid, myristic acid, pentadecanoic acid and hexadecenoic acid.
G6. The composition of one of Embodiments G1-G5, wherein the monoglyceride comprises a carboxylic acid selected from the group consisting of propionic acid, butyric acid, caproic acid, and lauric acid.
G7. The composition of any one of Embodiments G1-G6, wherein the monoglyceride comprises monolaurin, or is monolaurin.
G8. The composition of any one of Embodiments G1-G7, wherein the monoglyceride comprises a mixture of alpha-monoglycerides.
G9. The composition of any one of Embodiments G1-G8, wherein the monoglyceride is present at a level of from about 30% to about 70%, or from about 40% to about 60%, or from about 45% to about 55%.
G10. The composition of any one of Embodiments G1-G9, comprising a turmeric extract, wherein the turmeric extract comprises the curcuminoid.
G11. The composition of any one of Embodiments G1-G10, wherein the curcuminoid is a component of an extract, the extract being present in the composition at a level of from about 0.1% to about 5%, or from about 0.2% to about 2%, or from about 0.5% to about 1%.
G12. The composition of any one of Embodiments G1-G11, wherein the thymoquinone is present at a level of from about 0.01% about 0.2%.
G13. The composition of any one of Embodiments G1-G12, wherein the thymol is present at a level of from about 0.01% about 0.2%.
G14. The composition of any one of Embodiments G1-G13, wherein the carvacrol is present at a level of from about 0.01% about 0.2%.
G15. The composition of any one of Embodiments G1-G14, comprising a Monarda extract, wherein the Monarda extract comprises the thymoquinone, the thymol, and the carvacrol.
G16. The composition of Embodiment G15, wherein the Monarda extract is present at a level of from about 0.1% to about 5%, or from about 0.2% to about 2%, or from about 0.5% to about 1%.
G17. The composition of any one of Embodiments G1-G16, further comprising a carrier suitable for administration to a human or animal subject.
G18. The composition of Embodiment G17, wherein the carrier is operable for oral, nasal, or pulmonary administration.
G19. The method of any one of Embodiments G1-G18, wherein the subject is human and the viral-mediated disorder is associated with viruses of the family Coronaviridae (e.g., MERS-CoV, SARS-CoV, SARS-CoV-2), or viruses of the family Orthomyxoviridae (e.g., Influenza A, Influenza B).
G20. The method of any one of Embodiments G1-G18, wherein the subject is a livestock animal and the viral-mediated disorder is associated with Porcine Epidemic Diarrhea Virus, Transmissible Gastroenteritis, Porcine Respiratory Reproductive Syndrome, African Swine Fever Virus, Foot-and-Mouth Virus, Seneca Valley Virus, Influenza A, Avian Influenza H5N1, Rotavirus A, Rotavirus B, Rotavirus C, or Rotavirus D.
G21. The method of any one of Embodiments G1-G20, wherein the subject has pneumonia.
G22. The method of any one of Embodiments G1-G21, wherein the subject has severe acute respiratory syndrome.
G23. The method of any one of Embodiments G1-G22, wherein the method prevents or treats hypercytokinemia.
G24. The method of any one of Embodiments G1-G23, wherein the method mediates an adaptive immune response.

### Non-Limiting Discussion of Terminology

The headings (such as “Introduction” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The description and specific examples, while indicating embodiments of the technology, are intended for purposes of illustration only and are not intended to limit the scope of the technology. Equivalent changes, modifications and variations of specific embodiments, materials, compositions and methods may be made within the scope of the present technology, with substantially similar results. Moreover, recitation of multiple embodiments having stated features is not intended to exclude other embodiments having additional features, or other embodiments incorporating different combinations of the stated features. For example, a component which may be A, B, C, D or E, or combinations thereof, may also be defined, in some embodiments, to be A, B, C, or combinations thereof. Specific examples are provided for illustrative purposes of how to make and use the compositions and methods of this technology and, unless explicitly stated otherwise, are not intended to be a representation that given embodiments of this technology have, or have not, been made or tested.

As used herein, the words “prefer” or “preferable” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein. Further, as used herein the term “consisting essentially of” recited materials or components envisions embodiments “consisting of” the recited materials or components.

“A” and “an” as used herein indicate “at least one” of the item is present; a plurality of such items may be present, when possible. “About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters.

Unless otherwise stated herein, or evident in context, all percentages are by weight of composition.

As referred to herein, ranges are, unless specified otherwise, inclusive of endpoints and include disclosure of all distinct values and further divided ranges within the entire range. Thus, for example, a range of “from A to B” or “from about A to about B” is inclusive of A and of B. Further, the phrase “from about A to about B” includes variations in the values of A and B, which may be slightly less than A and slightly greater than B; the phrase may be read be “about A, from A to B, and about B.” The phase “less than about x” means about X or less than X. Similarly, the phrase “greater than about X” means about X or greater than X. Disclosure of values and ranges of values for specific parameters (such as temperatures, molecular weights, weight percentages, etc.) are not exclusive of other values and ranges of values useful herein.

It is also envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

1.-53. (canceled)

54. A composition for administration to a human or other animal subject, comprising
a monoglyceride;
a curcuminoid;
thymoquinone;
thymol;
carvacrol; and
p-cymene.

55. The composition of claim 54, wherein the monoglyceride comprises from about 30% to about 70%, of an alpha-monoglyceride selected from the group consisting of propionic acid, butyric acid, caproic acid, and lauric acid.

56. The composition of claim 55, wherein the monoglyceride comprises monolaurin.

57. The composition of claim 54, wherein the curcuminoid is a component of an extract, the extract being present in the composition at a level of from about 0.1% to about 5%, or from about 0.2% to about 2%, or from about 0.5% to about 1%.

58. The composition of claim 54, wherein the thymoquinone is present at a level of from about 0.01% about 0.2%.

59. The composition of claim 54, wherein the thymol is present at a level of from about 0.01% about 0.2%.

60. The composition of claim 54, wherein the carvacrol is present at a level of from about 0.01% about 0.2%.

61. The composition of claim 54, comprising from about 0.1% to about 5% of a Monarda extract, wherein the Monarda extract comprises the thymoquinone, the thymol, and the carvacrol.

62. A composition for nutritional support of a human or animal subject comprising a nutritionally safe and effective amount of a composition of claim 54.

63. An animal food composition comprising the composition of claim 62.

64. The composition of claim 63, in the form of a nutritional supplement or food additive.

65. A composition for administration to a human or other animal subject, comprising
a monoglyceride;
a curcuminoid; and
a Monarda extract comprising thymoquinone, thymol, carvacrol, and p-cymene.

66. The composition of claim 65, wherein the monoglyceride comprises monolaurin, or is monolaurin.

67. The composition of claim 65, in the form of a nutritional supplement or food additive.

68. A method for maintaining or improving a nutritional status of a human or animal subject having or at risk of having a viral-mediated disorder, comprising administering to the subject a composition comprising two or more of the following components:
a monoglyceride;
a curcuminoid;
thymoquinone;
thymol;
carvacrol; and
p-cymene.

69. The method of claim 68, wherein the composition comprises four or more of the components.

70. The method of claim 69, wherein the monoglyceride comprises monolaurin.

71. The method of claim 68, wherein the viral-mediated disorder is associated with viruses of the family Coronaviridae (e.g., MERS-CoV, SARS-CoV, SARS-CoV-2), or viruses of the family Orthomyxoviridae (e.g., Influenza A, Influenza B).

72. The method of claim 71, wherein the subject has severe acute respiratory syndrome.

73. The method of claim 68, wherein the viral-mediated disorder is associated with Porcine Epidemic Diarrhea Virus, Transmissible Gastroenteritis, Porcine Respiratory Reproductive Syndrome, African Swine Fever Virus, Foot-and-Mouth Virus, Seneca Valley Virus, Influenza A, Avian Influenza H5N1, Rotavirus A, Rotavirus B, Rotavirus C, or Rotavirus D.
